(19) United States
(12) Patent Application Publication
(10) Pub. No.: US 2009/0226368 A1
(43) Pub. Date: Sep. 10, 2009

(54) Androgen-Receptor (AR) Ligands for Use in the Treatment and Diagnosis of AR-Related Pathologies

(76) Inventors: Eyal Mishani, Mevasseret Zion (IL); Orit Jacobson, Jerusalem (IL); Einat Even-Sapir Weizer, Caesarea (IL)

Correspondence Address: THE NATH LAW GROUP, 112 South West Street, Alexandria, VA, 22314, US

(21) Appl. No.: 12/085,226

(22) PCT Filed: Nov. 21, 2006
(86) PCT No.: PCT/IL2006/001338

§ 371 Date: Mar. 17, 2009

Related U.S. Application Data
(60) Provisional application No. 60/738,053, filed on Nov. 21, 2005.

(51) Int. Cl. A61K 51/04; C07C 237/02; A61K 31/16; C12Q 1/02; A61P 5/00; A61P 5/26; A61P 15/00; A61P 35/00; A61P 9/10

(52) U.S. Cl. 424/1.65; 564/194; 514/626; 435/29

(57) ABSTRACT

The present invention discloses novel compounds and compositions suitable for the treatment of androgen related pathologies. The invention also provides diagnostic compositions for the diagnosis or monitoring of such androgen pathologies.

### FIELD OF THE INVENTION

This invention generally relates to compounds useful in the treatment and diagnosis of androgen-receptor related pathologies.

### BACKGROUND OF THE INVENTION

The androgen receptor (AR) is a member of the steroid/thyroid hormone receptor super-family that plays a critical role in the development and maintenance of male secondary sexual phenotype such as muscle, hair and bone mass, prostate growth and spermatogenesis. The AR is a cellular regulatory protein that upon androgen binding migrates into the nucleus, binds to specific DNA sequences referred to as the androgen response elements, and modulates the transcription of target genes.

The AR is also believed to be involved in prostate carcinogenesis and amplification of AR is present in most advanced prostate cancer specimens. Males who are castrated at a young age do not develop prostate cancer, a fact which may imply that androgens represent a risk factor for prostate cancer development. In addition, prostate-specific expression of an androgen receptor transgene in a transgenic mouse induces prostate intraepithelial neoplasia.

Prostate cancer is estimated to represent 30% of new cancer cases in U.S. males. Approximately 80-90% of prostate cancers are dependent on androgen at initial diagnosis and endocrine therapy of prostate cancer is directed toward the reduction of serum androgens and inhibition of AR. On the other hand, some very aggressive forms of prostate cancer were shown to have lost the expression of AR and are insensitive to inhibition of the AR. Testosterone and 5α-dihydrotestosterone (5α-DHT) are natural steroids that serve as natural ligands for the AR and which have been suggested as replacement therapy for androgen-deficiency.

Different ligands of the AR were reported and may generally be divided into two main structural groups, steroidal and nonsteroidal, with two different functionality classes, namely androgenic and antiandrogenic. Antiandrogens are used to counteract the undesirable actions of excessive androgens. Nonsteroidal antiandrogens, such as flutamide, nilutamide, and bicalutamide, were shown to bind exclusively to the AR and, therefore, exhibit little side-effects. These agents are advantageous over steroidal antiandrogens, such as megestrol acetate and cyproterone acetate, in terms of specificity, selectivity and pharmacokinetic properties and are successfully used clinically for the treatment of AR-dependent prostate cancer. Since the AR is a specific target for prostate cancer treatment and since loss of expression of AR has been observed in several aggressive tumors, it is critical to determine the role of AR in individual patients in order to guide and monitor treatment.

The standard tests for detecting prostate cancer include measurement of the prostate specific antigen (PSA) levels and digital rectal examination (DRE) assay, which includes prostate specimen collection by biopsy. PSA is regulated at the transcriptional level by the AR through androgen response elements in the promoter region of the gene. Nevertheless, the detection, using PSA levels, gives no information on the molecular characteristics of prostate cancer, whether it is AR-dependent or not. The biopsy has two main disadvantages: (1) it is an invasive procedure, and (2) tumors are very heterogenic, and the biopsy is limited to several areas in the tumor from which the samples were collected.

To date, imaging tools, including Positron Emission Tomography (PET) for diagnosing local recurrence and metastasis sites of prostate cancer are sub-optimal. PET, a nuclear medicine imaging technology, which allows the three-dimensional, quantitative determination of the distribution of radioactivity within the human body, is currently playing a major role in molecular imaging. This technology allows for the accurate measurement of radioactivity concentration in small volume elements in vivo as well as the ability to follow tracer kinetics. PET requires the administration to the subject of a molecule labeled with a positron emitting nuclide such as ¹⁵O, ¹³N, ¹¹C and ¹⁸F, having half-lives values of about 2, 10, 20 and 110 min, respectively.

Most prostate cancers are androgen dependent. Some very aggressive forms of prostate cancer were shown to have lost the expression of the AR. Although the AR is routinely targeted in endocrine treatment, the clinical outcome remains sub-optimal. Therefore, it is crucial to demonstrate the presence and activity of the AR in each case of prostate cancer, before and after treatment. While noninvasive PET has the potential to determine AR expression of tumor cells in vivo, fully optimized PET imaging agents are not yet available.

### SUMMARY OF THE INVENTION

The inventors of the present application have designed and synthesized a family of novel derivatives of hydroxyflutamide which have AR binding affinity that is similar to or higher than that of the currently used commercial drugs. Thus, these compounds are suitable both for the treatment of pathologies associated with the AR and for the diagnosis, monitoring of such pathologies. The novel compounds are AR-ligands, and thus are useful in binding to and activating the androgen receptor and as such may also be utilized in the study of the receptor.

Thus, in a first aspect of the present invention there is provided a compound of the general Formula I or a salt, ester, complex or enantiomer thereof:

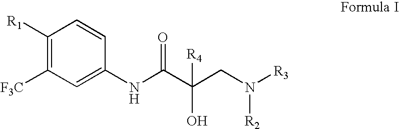

wherein

R1 is an electron withdrawing group;

R2 and R3, independently of each other, are each selected from H and a C1-C4 alkyl group being optionally substituted by at least one group selected from halogen, hydroxyl, alkyloxy, alkylthio, arylthio, alkoxy, alkylcarbonyl, carbonyl, alkoxycarbonyl, ester, amido, alkylamido, dialkylamido, aryl, benzyl, aryloxy, nitro, amino, alkyl or dialkylamino, carboxyl, or thio;

when R2 is H, R3 is a C1-C4 alkyl; and

R4 is a C1-C4 alkyl group being optionally substituted by at least one group selected from halogen, hydroxyl, alkoxy, alkylthio, alkylcarbonyl, carbonyl, alkoxycarbonyl, ester, amido, alkylamido, dialkylamido, aryl, benzyl, aryloxy, nitro, amino, alkyl or dialkylamino, carboxyl, and thio.

In one embodiment, in the compound of general Formula I, R2 or R3 is not a polyfluoroacylamido group, (e.g. perfluoroacylamido) or a C1-C4 substituted polyfluoroacylamido.

In another embodiment, in the compound of general Formula I, R1 is a radioisotope or comprising at least one radioisotope. Preferably, said radioisotope is selected from ¹¹C, ¹⁸F, ¹⁵O, ¹³N, ¹²⁴I or ⁷⁶Br.

In another embodiment, in the compound of general Formula I, R3 and/or R2, independently of each other is a C1-C4 group, as defined above, comprising at least one radioisotope. Preferably, said radioisotope is selected from ¹¹C, ¹⁸F, ¹⁵O, ¹³N, 124I or ⁷⁶Br.

In another embodiment, the compound of the general Formula I comprises at least two radioisotopes. The at least two radioisotopes may be bonded to a single atom, on the same functional group, on two different functional groups, etc. The at least two radioisotopes may or may not be the same.

In another embodiment, in the compound of the general Formula I R2 and R3, independently of each other is a C1-C4 alkyl being optionally substituted as above. Preferably, at least one atom of said C1-C4 alkyl group is a radioisotope, said atom being a carbon atom of the C1-C4 alkyl chain and/or any atom being substituted thereto.

In still a further embodiment, when R3 is —CH₃, said compound is of the formula II:

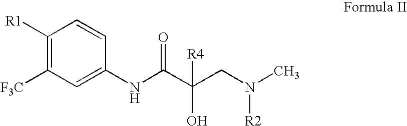

wherein each of R1, R2 and R4 is as defined hereinabove.

In another embodiment, in the compound of the Formula II, R4 is an unsubstituted C1-C4 alkyl.

In another embodiment, in the compound of Formula II, when R4 is —CH₃, said compound is of the Formula III:

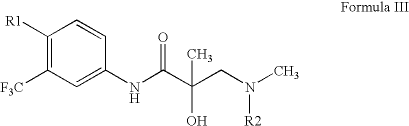

wherein each of R1 and R2 is as defined above.

In another embodiment, in the compound of Formula III, R2 is selected from H and a C1-C4 alkyl as defined above.

In another embodiment, when R2 is H said compound is of the Formula IV:

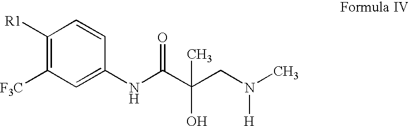

wherein R1 is as defined above.

In another embodiment, in the compound of Formula III, R2 is C1-C4 alkyl group, being optionally substituted by at least one group selected from halogen, hydroxyl, alkyloxy, alkylthio, alkoxy, alkylcarbonyl, carbonyl, alkoxycarbonyl, ester, amido, alkylamido, dialkylamido, aryl, benzyl, aryloxy, nitro, amino, alkyl or dialkylamino, carboxyl, and thio.

In yet another embodiment, in the compound of Formula III, R2 is an unsubstituted C1-C4 alkyl selected from methyl, ethyl, propyl, iso-propyl, butyl and t-butyl.

In yet another embodiment, in the compound of Formula III, R2 is a linear, unsubstituted C1-C4 alkyl selected from methyl, ethyl, propyl and butyl.

In another embodiment, said unsubstituted C1-C4 alkyl is methyl, and the compound is of the Formula V:

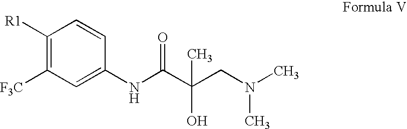

wherein R1 is as defined above.

In another embodiment, the compounds of the general Formula I, or the Formulas II, III, IV or V are radiolabeled each by at least one radioactive isotope.

In another embodiment, the compounds of the general Formula I, or the Formulas II, III, IV or V are “cold” compounds, namely not radioactively labeled.

In yet another embodiment, in the compound of the general Formula I or the Formulas II, III, IV, or V, R1 is selected from —CF₃, —CN, —NO₂, —F, —I, —Br, —CHO, —COO-alkyl, or —CO-alkyl.

In another embodiment, R1 is a radiolabeled group selected from —CF₃, —CN, —NO₂, —F, —I, —Br, —CHO, —COO-alkyl, and —CO-alkyl.

In another embodiment, R1 is an organic group selected from —CF₃, —CN, —CHO, —COO-alkyl, or —CO-alkyl.

In another embodiment, R1 is a group containing at least one halogen atom, said group being selected from —CF₃, —F, —I, and —Br.

In still another embodiment, R1 is a group containing at least one radioisotope of a halogen. Preferably, said at least one radioisotope is one or more of ¹⁸F, ¹²⁴I, and ⁷⁶Br.

In still another embodiment, R1 is selected from —F, —NO₂ or —CN. Each of said —F, —NO₂ or —CN may or may not be radiolabeled.

In another embodiment, in the compound of the Formula V, when R1 is —CN, said compound herein designated as Compound 1:

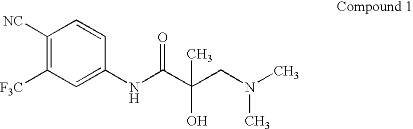

In another embodiment, in the compound of Formula V, when R1 is —NO₂, said compound herein designated as Compound 2:

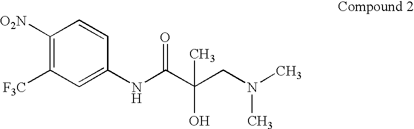

In yet another embodiment, in the compound of Formula V, when R1 is —F, said compound herein designated as Compound 3:

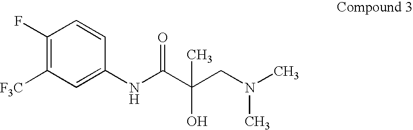

In yet another embodiment there is provided a radiolabeled derivative of each of Compounds 1, 2, and 3, said derivative containing at least one radioisotope.

In still another embodiment, there is provided the R-isomer of each of Compounds 1, 2, and 3.

In still another embodiment, there is provided the S-isomer of each of Compounds 1, 2, and 3.

In still another embodiment, there is provided a racemate of each of Compounds 1, 2, and 3.

The term “election withdrawing group” is art-recognized as a chemical substituent that withdraws electrons from the chemical group to which it is attached. Examples of electron withdrawing groups include, without limitation, —CF₃, halo (—Br, —Cl, —I, —F), —CN, —SO₃H, —CO₂H, —CO₂M, —CHO, —COM, —NO₂, and the like, wherein M is an alkyl of 1 to 6 carbon atoms or H.

The term “alkyl” refers to a saturated aliphatic hydrocarbon having between 1 and 6 carbon atoms, preferably between 1 and 4 carbon atoms, which may be arranged as a straight chain or branched chain, or as a cyclic group. The term “C1-C4 alkyl” or “an alkyl of 1 to 4 carbon atoms” refers to carbon chains having between 1 and 4 carbon atoms. These are, for example, methyl, ethyl, propyl, isobutyl, and butyl.

The alkyl group may be unsubstituted or substituted with one or more of a variety of groups selected from halogen, hydroxyl, alkyloxy, alkylthio, arylthio, alkoxy, alkylcarbonyl, carbonyl, alkoxycarbonyl, ester, amido, alkylamido, dialkylamido, aryl, benzyl, aryloxy, nitro, amino, alkyl or dialkylamino, carboxyl, thio, and others, each optionally being isotopically labeled. When substituted by a terminal group, the alkyl is an alkylene having between 1 and 6 carbon atoms. The alkyl or allylene group may contain one or more double or triple bonds as part of the hydrocarbon skeleton or appended as a substituent to said hydrocarbon skeleton or to any other substituent as disclosed above.

The term “aryl” as used herein refers to a group or part of a group having an aromatic system which may include a single ring or multiple aromatic rings fused or linked together where at least one part of the fused or linked rings forms the conjugated aromatic system e.g. having 6 to 14 carbon atoms. The aryl groups may for example include, but are not limited to, phenyl, naphthyl, biphenyl, anthryl, tetrahydronaphthyl, phenanthryl, indene, benzonaphthyl, fluorenyl, and carbazolyl.

The aryl group may be substituted by one or more substituents such as halogen, hydroxyl, alkyloxy, alkylthio, arylthio, alkoxy, alkylcarbonyl, carbonyl, alkoxycarbonyl, ester, amido, alkylamido, dialkylamido, benzyl, aryloxy, nitro, amino, alkyl or dialkylamino, carboxyl, thio, and others, each optionally being isotopically labeled.

The term “alkoxy” as used herein refers to the —O-(alkyl) group, where the point of attachment is through the oxygen-atom and the alkyl group is as defined hereinbefore.

The term “aryloxy” as used herein refers to the —O-(aryl) group, where the point of attachment is through the oxygen-atom and the aryl group is as defined hereinbefore.

The term “alkyloxy” includes hydroxyalkyl and as used herein refers to the -alkyl-OH group, where the point of attachment is through the alkyl group which is defined as hereinbefore. The term “alkylthio” refers similarly to -alkyl-SH.

The term “arylthio” as used herein refers to the —S-(aryl) group, where the point of attachment is through the sulfur-atom and the aryl group is as defined hereinbefore.

The term “alkylcarbonyl” as used herein refers to the —C═(O)-(alkyl) group, where the point of attachment is through the carbon-atom of the carbonyl moiety and the alkyl group is as defined hereinbefore.

The term “amino” refers to primary, secondary and tertiary amines where the point of attachment is through the nitrogen-atom. In case of the secondary or tertiary amines, the substituting groups on the nitrogen may be the same or different. In some embodiments, the amine group is a quaternary amine carrying a positive charge. In such embodiments, the charged amine is neutralized by a counter ion, as may be known to a person skilled in the art.

The term “halogen” or “halo” as used herein refers to —Cl, —Br, —F, or —I groups.

The term “ester” as used herein refers to a —C═(O)—O—, where the points of attachment are through both the C-atom and O-atom. One or both oxygen atoms of the ester group can be replaced with a sulfur atom, thereby forming a “thioester”, i.e., a —C═(O)—S—, —C═(S)—O— or —C═(S)—S— group. The term “carbonyl” refers to the group —C═O wherein the points of attachments are through the carbon atom. Such carbonyls may form ketones, i.e. a RRC═(O) group where the two R groups may or may not be the same, aldehydes, i.e. wherein in the group RRC═(O) one R is H and the other is a carbon substituents and the like. The term “carboxyl” refers to the group R—C═(O)—OH, when R may be an alkyl group as defined hereinabove.

The term “hydroxyl” refers to —OH, and “thio” refers to —SH.

The term “amido” refers to the group —C═(O)—NRR, wherein the points of attachments are through the carbon atom on one hand, and through the nitrogen atom on the other, wherein both R groups are H. When one group is H and the other is an alkyl, i.e., —C═(O)—NHR, the group refers to “alkylamido”. When both R5 are alkyls, the group refers to a “dialkylamido”.

The term “nitro” refers to the group —NO₂.

It should be understood that the compounds of the present invention contain each at least one chiral center which is the alpha-carbon bearing the group R4. The compounds of the invention may contain one or more additional chiral centers, and thus may exist in, and be isolated as, enantiomeric or diastereomeric forms, or as racemic mixtures. The present invention includes any possible enantiomers, diastereomers, racemates or mixtures, of any compound of the general Formula I. Where the herein-described processes for the preparation of the compounds of use in the invention give rise to mixtures of stereoisomers, these isomers may be separated by conventional techniques, such as preparative chromatography. The compounds may be prepared in racemic form, or individual enantiomers may be prepared either by enantiospecific synthesis or by chiral chromatographic separation of a racemate.

In one embodiment, the compounds of the invention are racemic mixtures of the R and S enantiomers. In another embodiment, the compounds are substantially pure R enantiomers. In another embodiment, the compounds are substantially pure S enantiomers. As used herein, the expression “substantially pure” is defined as greater than about 95% preponderance of one isomer.

The compounds of the present invention, namely those which fall within the general Formula I or any one of Formulas II to V, can be formulated into a composition, preferably pharmaceutical compositions, as neutralized pharmaceutically acceptable salt forms. Pharmaceutically acceptable salts include the acid addition salts (formed with the free amino groups), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Some of the compounds of the invention are acidic and they form salts with a pharmaceutical acceptable cation. Some of the compounds of this invention are basic and form salts with pharmaceutical acceptable anions. All such salts are within the scope of this invention and they can be prepared by conventional methods such as combining the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate.

The compounds of the invention may be prepared from a suitable precursor such as Compound 4 following a one-step or two-step procedure, as disclosed hereinnext. Thus, the present invention provides a method for the preparation of a compound of the general Formula I, said method comprising:

(i) condensing a suitable precursor molecule of a compound of general Formula I, with a mono-alkylamine (under conditions which allow condensation), thereby affording a mono-alkylamine derivative of the compound of Formula I, and

(ii) reacting said mono-alkylamine derivative with an alkylating agent to thereby afford a compound of the general Formula I.

In one embodiment, the said suitable precursor molecule of a compound of general Formula I is a compound of Formula V.

In another embodiment, the mono-alkylamine employed in step (i) is methylamine.

In another embodiment, the alkylating agent of step (ii) is an alkylhalide selected from alkylbromide, alkylchloride and alkyliodide. Preferably, the alkylhalide is allyliodide, more preferably methyl iodide.

In another embodiment, one or both of said allylamine and alkylating agent are radioactively labeled.

The mono-methyl derivative afforded in step (i) of the above method is herein designated as Compound 4, when R1 is —CN, Compound 5, when R1 is —NO₂ and Compound 6, when R1 is —F.

Thus, this method allows the preparation of the Compounds 1, 2, 3, 4, 5 and 6.

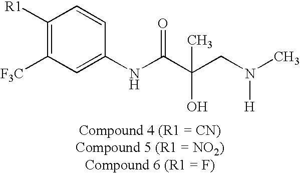

This method may be applied to afford a radiolabeled end product. If radiolabeling is required at the electron withdrawing group R1, the precursor molecule may be pre-labeled by methods known to a person skilled in the art or by any of the methods disclosed herein. If, on the other hand, labeling is required on the R3 group, the above method may utilize radiolabeled alkylamine.

The expression “conditions which allow condensation” refers to a set of conditions which would be familiar to a person skilled in the art of organic synthesis and which would not require undue experimentation to determine. The presence of the end-product of such a condensation reaction or any other transformation reaction may be tested by utilizing one or more of known spectroscopic or chromatographic methods, such as TLC, UV, HPLC, GC-MS, NMR and others known to a person versed in the art.

In another approach to the synthesis of a dialkyl, e.g., a dimethyl end product such as Compounds 1 to 3, the method for the preparation of a compound of general Formula I comprises contacting a suitable precursor molecule of the Formula VI with dialkyl amine. The two alkyl groups of the dialkylamine may or may not be the same, and may or may not be radioactively labeled. If labeled, the dialkylamine may be di- or mono-labeled by ¹¹C or ¹³N. This method affords Compounds 1, 2 and 3 in one step.

The methods of preparation claimed and disclosed are merely examples of the various methodologies, which may be used for the preparation of the compounds of general Formula I.

In some embodiments, the method of preparation further comprises the step of enantiomeric resolution in order to obtain a single isomer or enrich a racemic mixture with one isomer. Alternatively, the desired isomer may be synthesized enantiospecifically, thus affording said isomer without necessitating further separation.

In yet another aspect of the invention there is provided a composition comprising the compound of general Formula I or a salt, ester, complex or enantiomer thereof.

In one embodiment, the composition is a pharmaceutical composition. Preferably, the pharmaceutical composition comprises at least one non-labeled compound of the general Formula I.

In another embodiment, the composition is a diagnostic composition. Preferably, the diagnostic composition, as will be discussed next, comprises at least one radioactively labeled compound of the general Formula I.

Therapeutic compositions as well as diagnostic compositions may be used for human or animal purposes. The term “animal” refers to a non-human animal.

The pharmaceutical composition may comprise at least one compound of the general Formula I and at least one further pharmaceutically acceptable carrier, diluent or excipient. The composition may comprise a single compound of the general Formula I, which may or may not be radioactively labeled; two or more compounds of the general Formula I, for example one being “cold” and the other radioactively labeled, and so on.

The pharmaceutical composition of the invention may be used for the treatment of various androgen-related pathologies, i.e. diseases or disorders. Such pathologies may be selected from (a) hormone related conditions, for example conditions associated with androgen decline in an aging male such as fatigue, depression, decreased libido, sexual dysfunction, anemia, obesity and the like; (b) androgen decline in females and males such as sexual dysfunction, hypogonadism, osteopenia, obesity, alternation in cognition and mood and others; (c) chronic muscular wasting; (d) androgen related neoplastic diseases; (e) prostate cancer; (f) benign prostate hyperplasia; (g) cancers of female sexual organs such as breast cancers, uterine cancer and ovarian cancer.

Preferably, the AR-related pathology is related to a disease or disorder selected from osteoporosis, periodontal disease, bone fracture, bone damage following bone reconstructive surgery, sarcopenia, frailty, aging skin, male hypogonadism, post-menopausal symptoms in women, female sexual dysfunction, atherosclerosis, hypercholesterolemia, hyperlipidemia, aplastic anemia and other hematopoietic disorders, pancreatic cancer, renal cancer, prostate cancer, breast cancer, hepatocellular carcinoma, glioma, meningioma, arthritis and joint repair.

In a preferred embodiment, the pathology is a disease or disorder of the prostate.

Thus, the present invention provides a pharmaceutical composition for the treatment of disease or disorder of the prostate, said composition comprising at least one compound of general Formula I or a salt, ester, complex or enantiomer thereof. In one preferred case, said disease or disorder is prostate cancer.

As used herein, “pharmaceutical composition” means therapeutically effective amounts of a compound of the present invention, together with suitable diluents, preservatives, solubilizers, emulsifiers, adjuvant and/or carriers. Such compositions are liquids or lyophilized or otherwise dried formulations and include diluents of various buffer content (e.g.; Tris-HCL, acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils).

Also comprehended by the invention are particulate compositions coated with polymers (e.g., poloxamers or poloxamines). Other embodiments of the compositions of the invention incorporate particulate forms, protective coatings, protease inhibitors or permeation enhancers for various routes of administration, including parenteral, pulmonary, nasal and oral. In one embodiment the pharmaceutical composition is administered parenterally, paracancerally, transmucosally, transdermally, intramuscularly, intravenously, intradermally, subcutaneously, intraperitonealy, intraventricularly, intracranially and intratumorally.

In yet another embodiment, the pharmaceutical composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989).

In another embodiment, polymeric materials can be used.

In yet another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984)). Preferably, a controlled release device is introduced into a subject in proximity to the site of inappropriate immune activation or a tumor. Other controlled release systems are discussed in the review by Langer (Science 249:1527-1533 (1990)).

According to another aspect of the present invention, the use of a compound of the invention for the preparation of a pharmaceutical composition for treatment of androgen receptor related pathology in a patient (i.e. suffering from an androgen dependent condition) is provided.

According to another aspect of the present invention, the use of a compound of the invention for the preparation of a pharmaceutical composition for treating a subject having prostate cancer is provided. In one embodiment, said compound is selective for androgen or testosterone receptor.

The compounds of the invention have affinity for the androgen receptor and will cause a biological effect by binding to the receptor. In selected embodiments they will act as partial agonists or antagonists, full agonists or antagonist, or tissue selective agonists or antagonist. Preferably, the compounds of the invention are partial agonists or antagonists to the androgen receptor. As androgen receptor modulators, the compounds can be used to treat, or alleviate, conditions associated with inappropriate activation of the androgen receptor. Examples of such conditions for antagonists include, but are not limited to, acne, excess sebum secretion, androgenic alopecia, hormone dependant cancers such as prostrate cancer, and hirsutism. Those compounds which are partial agonists, full agonists, or tissue selective agonists can be used to treat osteoporosis, hypogonadism, anemia, or to stimulate increases in muscle mass, especially in wasting diseases.

As used herein, the term “agonist” refers to a compound of the invention that binds to the androgen receptor and mimics the effect of the natural ligand. An “antagonist” is a compound that prevents or inhibits the effect of the natural ligand. The term “tissue selective agonist” refers to a compound that mimics the action of the natural ligand in some tissues but not in others.

In yet another aspect of the invention, there is provided a method for the treatment of androgen related disease or disorder, said method comprising administering to a subject in need thereof an effective amount of a composition comprising at least one compound of general Formula I or a salt, ester, complex or enantiomer thereof.

The term “treatment” as used herein refers to the administering of a therapeutic amount of the composition of the present invention which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above. The “effective amount” for purposes disclosed herein is determined by such considerations as may be known in the art. The amount must be effective to achieve the desired therapeutic effect as described above, depending, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the affinity of the ligand to the receptor, its distribution profile within the body, a variety of pharmacological parameters such as half life in the body, on undesired side effects, if any, on factors such as age and gender, etc.

The pharmaceutical compositions of the invention may comprise additionally any other suitable substances such as other therapeutically useful substances, diagnostically useful substances, pharmaceutically acceptable carriers or the like.

In a further embodiment of the invention, the compounds of Formula I can be co-administered with other compounds to further enhance their activity, or to minimize potential side effects.

According to another aspect of the present invention, a method is provided for binding the compounds of the present invention to an androgen receptor by contacting the receptor with a compound of the invention under conditions effective to cause said compound to bind the androgen receptor. The binding of the compound is reversible or irreversible, preferably reversible.

In yet another aspect of the invention, the compound of general Formula I or a salt, ester, complex or enantiomer thereof is a radioactively labeled compound of the general Formula I. The radiolabeling may be by one or more radioactive isotopes of, e.g., C, O, N, I, F or Br. In one embodiment, the compound is labeled with a single isotope. In another embodiment, the compound is labeled with two different isotopes, one of which preferably being ¹¹C.

Thus, the present invention further provides a radiopharmaceutical of the general Formula I or a salt, ester, complex or enantiomer thereof. In one embodiment, said radiopharmaceutical is used as an imaging agent for molecular imaging of at least one disease or disorder.

In one embodiment, said disease or disorder is associated with androgen related pathologies, as defined hereinbefore.

In another embodiment, the compounds of general Formula I are used for the detection of prostate cancer.

The radiopharmaceutical may be used in the preparation of diagnostic compositions and kits suitable for use in the detection of such diseases and disorders associated with AR pathologies. In a preferred embodiment, said pathologies are cancer related.

In preferred embodiments, the radioactive agents of the present invention are used as imaging agents for PET imaging.

Thus, the present invention further provides a method for imaging a tissue containing androgen receptor, said method comprising:

(i) contacting said tissue containing androgen receptor with an effective amount of a radiolabeled compound according to Formula I under conditions which allow binding between said compound and the receptor;

(ii) subjecting the tissue to PET imaging; and

(iii) detecting regions of said tissue in which the radiolabeled compound has bound to an androgen receptor.

As stated above, the PET detection methods produce images of diseased glands which have absorbed a radioactive marker, i.e. the radiopharmaceutical compounds of the invention. Such detection or diagnosis may be performed at any time within the first several hours after a patient has been administered, typically injected, with the radiopharmaceutical. During the process, images are taken of the site of the disease. The resulting images may, depending on the instrument employed, be viewed separately, or assimilated to provide a three dimensional picture.

In some embodiments, the imaging using any applicable method may be for the diagnosis, staging, re-staging or monitoring of the androgen-related pathology.

In one preferred embodiment, diagnosis means the primary and secondary detection of a disease in an organism or a sample. In another embodiment, the diagnostic method may be used to determine the minimal residual disease of a tumor after primary therapy.

There is also contemplated in the present invention, a method of diagnosis of carcinomas and their precursor lesions, which may be applied in routine screening tests for preventive aspects in order to detect a certain disease at an early stage of the onset of the disorder. For the purpose of such an early detection, samples obtained by minimally invasive methods such as blood samples, stool samples, sputum samples, nipple aspirates or those collected during the colonoscopy, bronchioscopy, or other such methods, may be employed.

Accordingly, the invention also provides a method of monitoring treatment of a subject. The method involves administering to a subject having cancer cells or cells associated with an angiogenesis function disorder a compound described above and measuring the survival of the cells, the growth of the tumor, or a combination thereof using PET imaging. The subject may be suffering from leukemia, non-small cell lung cancer, colon cancer, CNS cancer, melanoma, ovarian cancer, breast cancer, renal cancer, hepatocellular carcinoma, glioma, meningioma, or prostate cancer. The subject may be an animal, e.g., a mouse, and the cells may be xenografted human cells. Preferably, the subject is a human.

This method may also be utilized for monitoring treatment of a subject ex vivo.

The radiopharmaceuticals of the invention may be administered in pharmaceutically acceptable forms, in compositions which may additionally comprise any other suitable substances such as other diagnostically useful substances, therapeutically useful substances, carriers as defined above or the like.

Also provided are therapeutic or diagnostic kits comprising the compounds of the invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains. The following examples and biological data is presented in order to further illustrate the invention. This disclosure should not be construed as limiting the invention in any manner.

### BRIEF DESCRIPTION OF THE FIGURES

In order to understand the invention and to see how it may be carried out in practice, numerous examples are provided and should be considered as non-limiting of the invention as disclosed.

FIGS. 1A-E show the blood stability chromatograms of ¹⁸F-labeled (R)-Compound 3;

FIG. 2 shows the results of the blood stability experiments of ¹¹C-labeled (R)-Compound 3;

FIG. 3 presents a PET/CT image of normal Lewis male rat 40 minutes post injection of 300 μCi of ¹¹C-labeled (R)-Compound 3;

FIGS. 4A-B demonstrates the antagonist characteristics of ¹⁸F-labeled (R)-Compound 3 after 48 hours and after 72 hours.

### DETAILED DESCRIPTION OF THE INVENTION

It has been previously suggested that electron-withdrawing groups located at the aromatic ring, coupled to a branched alkyl group a to the amide were required for binding and functional activity of efficient androgen ligands. These electron-withdrawing groups induce the withdrawal of electrons in the molecule towards the aromatic ring which leads to the rapid deprotonation of the hydroxyl group or the amide group or both, for example in the case of hydroxyflutamide, which results in the decomposition of the molecule and intra nucleophilic attack forming a lactam or an epoxide.

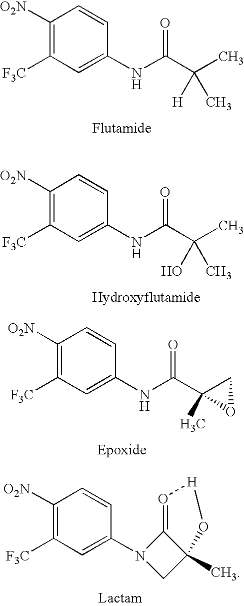

¹⁸F-labeled nonsteroidial antiandrogen compounds, ¹⁸F-labeled flutamide derivatives, (R)-3-Bromo-N-(4-fluoro-3-(trifluoromethyl)phenyl)-2-hydroxy-2-methyl-propanamide, herein designated R—[¹⁸F]-1 and N-(4-fluoro-3-(trifluoromethyl)phenyl)-2-hydroxy-2-methylpropanamide, herein designated R—[¹⁸F]-2 have been previously prepared by the inventors of the present invention (Jacobson O., et al., Bioorg Med. Chem. 2005, 13(22), 6195-6205). Although similar compounds labeled with ⁷⁶Br have also been previously reported they exhibited very low in-vivo stability, probably due to de-bromination.

It was surprisingly discovered in the course of the study which lead to the present invention, that improved in-vivo potency may be achieved by utilizing nonsterodial androgen receptor ligands such as those disclosed herein. Radiolabeling of these novel compounds with ¹¹C and/or ¹⁸F allowed the use of these compounds in the diagnosis of related pathologies.

These molecules containing the functional electron withdrawing groups at the aromatic moiety were shown to be essential for AR binding. In order to stabilize the electron distribution formed by the strong electron withdrawing effect directed towards the aromatic ring, an electron rich group, such as dimethylamine, was added at the opposite side of the pole, i.e., methyl group, replacing one of the hydrogens and forming chiral center. This stabilization of the electron distribution within the basic structure of flutamide resulted in a better in-vivo bioavailability.

The (R) enantiomers of Compounds 1-3 exhibited better in-vitro results in AR binding assay than the commercial drugs flutamide and bicalutamide, which are used today clinically. In order to achieve greater potency, the synthesis of the target ligands was carried out regio-selectively using D-proline to yield the R enantiomers.

Compounds 1-3 as well as Compounds 4-6 of the present invention may be used for the preparation of pharmaceutical compositions for the treatment of AR-related pathologies or diagnostic compositions for diagnosing and monitoring such pathologies. The compositions of the invention may comprise in addition to the active agent, namely at least one compound of the invention, any other suitable substances such as other therapeutically useful substances, diagnostically useful substances, pharmaceutically acceptable carriers or the like.

The pharmaceutically acceptable “carriers” described herein, for example, vehicles, adjuvants, excipients, or diluents, are well-known to those who are skilled in the art and are readily available to the public. It is preferred that the pharmaceutically acceptable carrier be one which is chemically inert to the active compounds and one which has no detrimental side effects or toxicity under the conditions of use. The choice of carrier will be determined in part by the particular active agent, as well as by the particular method used to administer the composition. Accordingly, there is a wide variety of suitable formulations of the pharmaceutical composition of the present invention.

The compositions may be administered to the subject in need thereof by any method known to a person skilled in the art. Non-limiting examples of such methods are: oral, parenteral, paracanceral, transdermal, transmucosal, intravenous, intraperitonal, intramuscular, intracranial, intravaginal, intratumoral or via inhalation. The composition of the invention may additionally be administered topically, as a slow-release formulation, in a liposome or as part of any carrier known to a person skilled in the art.

Formulations suitable for oral administration may consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodiumk talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

The compounds of the present invention, alone or in combination with other suitable components may also be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations, may include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and ally pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopriopionates, and 2-alkyl-imidazoline quaternary ammonium salts, and (3) mixtures thereof.

Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations may be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The compounds of the present invention may also be made into injectable formulations. The requirements for effective pharmaceutical carriers for injectable compositions are well known to those of ordinary skill in the art. See Pharmaceutics and Pharmacy Practice, J.B. Lippincott Co., Philadelphia, Pa., Banker and Chalmers, eds., pages 238-250 (1982), and ASHP Handbook on Injectable Drugs, Toissel, 4^{th} ed., pages 622-630 (1986).

Additionally, the compounds of the present invention may be made into suppositories by mixing with a variety of bases, such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

According to another aspect of the present invention, a method is provided for binding the compounds of the present invention to an androgen receptor by contacting the receptor with a compound of the invention under conditions effective to cause said compound to bind the androgen receptor. The binding of the compound is reversible or irreversible, preferably reversible.

The compound of general Formula I or a salt, ester, complex or enantiomer thereof may be a radioactively labeled compound. The radiolabeling may be by one or more radioactive isotopes of, e.g., C, O, N, I, F or Br. The compound may be labeled with a single isotope or with e.g., two different isotopes, one of which preferably being ¹¹C or ¹⁸F.

As such, each of the compounds of the invention may be a radiopharmaceutical suitable as an imaging agent for molecular imaging of at least one disease or disorder. The radiopharmaceutical may be used in the preparation of diagnostic compositions and kits suitable for use in the detection of such diseases and disorders associated with AR pathologies. In a preferred embodiment, said pathologies are cancer related.

Generally speaking, any methods of detection of the labeled molecules may be utilized in the course of the diagnosis of cancer-related pathologies and cancer-precursor lesions. Diagnosis may, for example, comprise the detection of cells or tissues affected by abnormal growth by any method known to a person skilled in the art. One such method is Positron Emission Tomography (PET).

PET imaging is a tomographic nuclear imaging technique that uses radioactive tracer molecules that emit positrons. When a positron meets an electron, they both are annihilated and the result is a release of energy in form of gamma rays, which are detected by the PET scanner. By employing natural substances that are used by the body as tracer molecules, PET does not only provide information about structures in the body but also information about the physiological function of the body or certain areas therein. Gamma radiation produced from a positron-emitting atom such as a fluorine atom is detected by the PET scanner and shows the metabolism of the compound bearing said atom in certain areas or tissues of the body, e.g. in the brain or the heart.

Scanning consists of either a dynamic series or a static image obtained after an interval during which the radioactive molecule enters the biochemical process of interest. The scanner detects the spatial and temporal distribution of the tracer molecule. PET also is a quantitative imaging method allowing the measurement of regional concentrations of the radioactive tracer molecule.

Commonly used radionuclides in PET tracers are ¹¹C, ¹⁸F, ¹⁵O, ¹³N, ¹²⁴I or ⁷⁶Br. Recently, new PET tracers were produced that are based on radiolabeled metal complexes comprising a bifunctional chelating agent and a radiometal. Bifunctional chelating agents are chelating agents that coordinate to a metal ion and are linked to a targeting molecule that will bind to a target site in the patient's body. Such a targeting molecule may be any compound that binds to a certain receptor, probably associated with a certain area in the body or with a certain disease. The advantage of such complexes is that the bifunctional chelating agents may be labeled with a variety of radiometals such as, and without being limited thereto, ⁶⁸Ga, ²¹³Bi or ⁸⁶Y.

Currently, the molecular imaging diagnosis of prostate cancer using PET is done exclusively by ¹¹C-choline, which is not a specific marker and cannot monitor and guide future treatment. Diagnosis of prostate cancer using the gold standard tracer ¹⁸F-FDG cannot be performed due to the high accumulation of free ¹⁸F-fluoride in the urine bladder which masks the prostatic bed. Such deficiencies may be overcome by using labeled Compounds 1-3, with ¹¹C on one of the methyl groups of the dimethylamine group, or with ¹⁸F on the aromatic ring. Such modifications have a better potential not only for the specific diagnosis of AR-dependent prostate cancer, but also for monitoring and guiding further treatment.

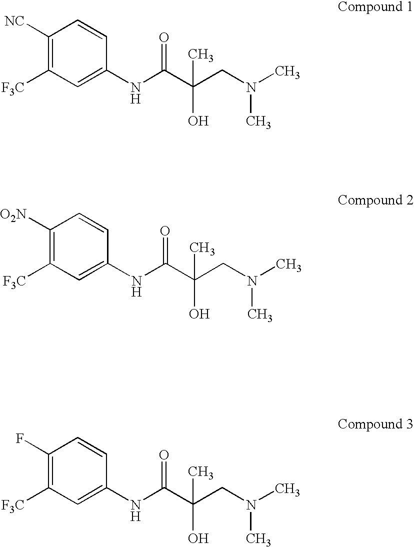

In order to measure the affinity of the compounds to the A, the compounds of the general Formula I, referred herein also as the AR ligands were tested in RBA (Relative Binding Affinity) assay, which is a competitive binding assay with ³H—R1881, a high affinity AR binding ligand. As can be seen from Table 1 below, (R)-Compound had an RBA of 0.030% which is similar to the RBA of hydroxyflutamide and bicalutamide and superior to flutamide and bicalutamide.

TABLE 1
A comparison study of the affinity of various AR ligands to the
AR receptor

| Compound | RBA (%) |
|---|---|
| R1881 | 100 |
| R-Bicalutamide | 0.044 |
| R,S-Bicalutamide | 0.037 |
| Flutamide | 0.001 |
| Hydroxyflutamide | 0.027 |
| 3-bromo hydroxyflutamide | 1.9 |
| (R)-F-1 | 0.057 |
| (R)-F-2 | 0.027 |
| (R)-Compound 1 | 0.030 |
| (R)-Compound 2 | 0.036 |
| (R)-Compound 3 | 0.029 |

There are two approaches to label Compounds 1-3 with carbon-11. As shown in Scheme 1, the first approach involved a reaction of labeled methyl-iodide with the monomethylamine precursors designated Compounds 4, 5 and 6.

The second approach, shown in Scheme 2, involved the reaction of novel labeled dimethylamine directly with a suitable precursor molecule of the Formula VI, wherein R1 is as defined above and X is a suitable leaving group as known to a person skilled in the art (e.g., X=halogen atom, sulfonate groups, etc). Labeling with fluorine-18 involved a three-step radio-synthesis which is shown in Scheme 3. This radio synthesis involved as a first step substitution of a nitro group ortho to the trifluoromethyl group and para to the second nitro group (compound 10), with ¹⁸F via an S_{N}2 nucleophilic displacement to afford compound 11. In the second step, the remaining nitro group was reduced to the amine, which was next coupled with an acid chloride such as compound 9, thereby affording the labeled forms of Compounds 1 to 3.

It should be noted that Schemes 1 and 2 are exemplary procedures only, given in order to present a clear and accurate description of the methods of preparation of the novel compounds of the invention. For example, compounds of Formula VI, as shown in these figures, may have any leaving group. While the bromide atom is a good leaving group in such systems, it is shown herein for illustrative purposes only and thus should not be considered limiting.

### Example 1

### Synthesis of (R)-Compound 1-(R)-3-Dimethyl-N-(4-cyano-3-trifluoromethyl-phenyl)-2-hydroxy-2-methyl-propionamide

Dimethylamine (2 mol/L in THF, 9 mL) was stirred and cooled in an ice bath. A solution of a (R)-compound of Formula VI wherein X═Br and R1=—CN (300 mg, 0.854 mmol) in THF (5 mL) was added drop-wise, and the reaction was stirred for 2 h. Then, ethyl acetate (30 mL) and saturated NaHCO₃ (30 mL) were added. The layers were separated, and the organic layer was washed with brine, dried (MgSO₄), and evaporated. The residual oil was purified by flash chromatography on silica gel (MeOH—CH₂Cl₂, 5:95) to give 120 mg (45%) of (R)-Compound 1 as a white solid.

MS (m/z) 316 (MH⁺);

¹H NMR (300 MHz, CDCl₃): δ 1.50 (s, 3H), 2.28 (s, 6H), 2.45 (d, 1H, J=3.0 Hz), 3.0 (d, 1H, J=3.0 Hz) 7.78 (d, 1H, J=6.0 Hz), 7.93 (dd, 1H, J=1.5, 6.0 Hz), 8.01 (d, 1H, J=1.5 Hz), 9.47 (s, 1H);

Elementary analysis: C=51.87%, H=5.28%, N=12.96%.

### Example 2

### Synthesis of (R)-Compound 2-(R)-3-Dimethyl-2-hydroxy-2-methyl-N-(4-nitro-3-trifluoromethyl-phenyl)-propionamide

(R)-Compound 2 was prepared similarly to (R)-Compound 1, as described in Example 1, using a (R)-compound of Formula VI with X=Br and R1=—NO₂.

### Example 3

### Synthesis of (R)-Compound 3-(R)-3-Dimethyl-N-(4-fluoro-3-(trifluoromethyl)phenyl)-2-hydroxy-2-methyl-propanamide

(R)-Compound 3 was prepared similarly to (R)-Compound 1, as described in Example 1, using a (R)-compound of Formula VI with X=Br and R1=F.

### Example 4

### Labeling with carbon-11

Two different approaches were employed to label the compounds of the invention with ¹¹C:

### (a) Direct Approach—Via Labeled Dimethylamine (as Shown in Scheme 2)

¹¹C-MeI was prepared according to known procedures. [¹¹C]CO₂ (37 GBq, 1000mCi) was trapped at −160° C. The temperature of the cooling trap was increased to −50° C., and the activity was transferred by a stream of argon (40 mL/min) into reactor 1 containing 300 μL of 0.25N LiAlH₄ in THF at −50° C.

After 90 seconds, the solvent was removed under reduced pressure. In this manner, more than 80% of the activity was recovered. The reactor temperature was increased to 160° C., HI was added, and [¹¹C]MeI was distilled (argon flow of 15 mL/min.) through a NaOH column to a second reactor, containing 0.2 mL of monomethylamine 2 mol/L in THF with 50 μL of dry DMSO at −20° C. At the end of a 1-minute distillation step, an average of 550 mCi (n=5) was trapped in the second reactor. The reactor was sealed, and heated to 45° C. for 5 minutes. At the end of the 5-minute reaction, the reactor was cooled to −10° C. and a solution of compound 4a-c (30 mg) in THF (300 μL) with 6 μL of N,N-diisopropylamine was added. The reactor was sealed, and heated to 25° C. for 12 minutes. At the end of the 12 minutes, the reactor was heated to 80° C. and THF was removed under flow of argon. The mixture was cooled to 40° C., 0.6 mL of HPLC solvent (20% H₂O, 80% CH₃CN) were added, and the crude product (average of 100 mCi (n=5)) was automatically injected to the HPLC [Bischoff Nucleosil 100-7-C18 reverse phase preparative column (7 μm, 250×16 mm), flow rate of 9 mL/min]. The labeled product was collected in a flask containing 60 μL of 1 mol/L NaOH in 85 mL of water. The solution was passed through a 2×C-18 cartridge (Waters Sep-Pak Plus, preactivated with 10 mL EtOH and 20 mL of sterile water), and the cartridge was washed with 4 mL of sterile water. The products were eluted with 0.75 mL of EtOH, followed by 4.25 mL of saline, and collected into the product vial after a total radiosynthesis time of 50 minutes. Identification of the products and of chemical and radiochemical purities was determined by reverse-phase HPLC C18 analytical column (40% CH₃CN, 60% H₂O, flow rate of 1 mL/min).

### (b) Second Approach—Monomethylamine (as Shown in Scheme 1)

The radiochemical synthesis was done in a similar manner as with the dimethylamine. After the formation of labeled methyl iodine in the first reactor, it was distilled through the NaOH column to a second reactor containing the precursor which was prepared as follows:

Methylamine (2 mol/L in THF, 1 mL) was added to a solution of a compound of Formula VI (12 mg) in dry DMSO (70 μL), at 0° C. After a 15-minute reaction, 0.015 mol/L NaOH (10 mL) were added. The solution was passed through 2×C-18 cartridges (Waters Sep-Pak Plus, Massachusetts, USA), pre-activated with 10 mL EtOH and 20 mL of sterile water), and the cartridges were dried under nitrogen stream. The product was eluted with 4 mL of THF, and dried with Na₂SO₄. Filtration with a 0.45 μm filter and THF evaporation under reduced pressure gave crude products designated herein as Compounds 4-6, which were used for the radiolabeling without any further purification.

### Example 5

### Labeling with Fluorine-18

K[¹⁸F]F-Kryptofix 2.2.2 complex—The [¹⁸F] ion was produced by the ¹⁸O(p,n) ¹⁸F nuclear reaction on 2 mL enriched [¹⁸O]water (95% isotopic purity) as a target at the Hadassah-Hebrew University IBA 18/9 Cyclotron and then transferred into a fluorination module by a flow of argon. After trapping, it was loaded onto an anion exchange column, dried, eluted with 1 mL of K₂CO₃ solution (2.76 mg/mL), and transferred to the reactor.

Reactive organic [¹⁸F] ion was prepared by adding 50-100 μL of the K[¹⁸F]F solution to Kryptofix 2.2.2 (1.0 mg, 2.7 lμmol) in acetonitrile. Azeotropic removal of water and acetonitrile was achieved by heating under a stream of nitrogen. As shown in Scheme 3, dried K[¹⁸F]F-Kryptofix 2.2.2 complex was then dissolved in 300 μL anhydrous DMSO for use in the radiolabeling. The K[¹⁸F]F-Kryptofix 2.2.2 complex in DMSO (300 μL) was added to compound 10 (2-3 mg, 8-13 μmol) in a screw-cap test tube (8 mL). The tube was capped, shaken and heated in the microwave for 3.5 min. After cooling to ambient temperature in a water bath, the vial content was diluted with 10 mL of water and loaded onto an activated (EtOH) and equilibrated (water) C18 Sep-Pak cartridge (classic, short body). The cartridge was washed with water (10 mL) and [¹⁸F]-11 was eluted with EtOH (2 mL) into a small glass test tube. [¹⁸F]-11 was analyzed using HPLC and compared to cold standards (Sigma) by co-injection; this analysis indicated that the nitro group at the ortho to the trifluoromethyl group was substituted exclusively. The reduction vessel was prepared by adding to a V-vial (5 mL), sequentially, a few borosilicate glass beads, EtOH-water (100 μL, 4:1), Raney nickel (50% slurry in water, 250 μL), and hydrazine monohydrate (60 μL, 1.2 μmol). After capping with a septum-equipped screw cap (vented with a large diameter needle), the vial was shaken and placed in a 55° C. heating block for 5 min. The solution of [¹⁸F]-11 in ethanol was diluted with water (0.5 mL) and was added slowly to the reduction vessel. After 5 min, an additional portion of hydrazine hydrate (40 μL, 0.8 μmol) was added to the reaction vessel. The reaction was stirred for an additional 10 min, and then the vessel was cooled to ambient temperature in a water bath and the vial content was filtered through a 0.45 mm filter (polypropylene) into another glass test tube. [¹⁸F]-12 was analyzed using HPLC and compared to cold standard (Sigma) by co-injection. To the filtered solution of [¹⁸F]-12, water (10 mL) was added and loaded onto an activated (EtOH) and equilibrated (water) C18 Sep-Pak cartridge (classic, short body). The cartridge was washed with dry dimethylacetamide (0.7 mL) into a 10 mL flask, which contained compound 9 under nitrogen at 0° C. The reaction solution was stirred for 0.5 h in an ice bath and for an additional 1 h at room temperature. The solution was filtered through a 0.2 μm filter (nylon) and injected onto the reverse phase HPLC (semi-preparative column). [¹⁸F]-labeled Compound 3 was analyzed by analytical reversed phase HPLC.

### Example 6

### Relative Binding Affinity (RBA)

RBA of androgens were measured against [³H]R1881 in human AR recombinant (Pan-Vera). RBA values of the standards are 100 by definition (Brandes S. J., et al., Molecular Pharmacology; 1987; 32:391-400).

### Example 7

### Blood Stability Tests

Blood was taken in EDTA tubes in order to prevent coagulation. 550 μl of ¹⁸F-labeled (R)-Compound 3 was added to 1 ml of blood, and put in a rotating incubator at 37° C. The incubation times were 0, 30, 60 and 90 minutes. After the incubation, the blood was centrifuged for 5 minutes at 3000 rpm. The plasma was centrifuged again for 5 minutes at 10600 g and the pellet was discarded. 400 μl of the resulting supernatant was added to 800 μl of 30% THF/CH₃CN, vortexed for 30 seconds and centrifuged for 5 minute at 10600 g. The supernatant was filtered through 0.45 μm filter and injected to HPLC column. The column was eluted with a mixture of acetate buffer (pH 3.8) and acetonitrile (35:65) at flow rate of 3 ml/min. The HPLC eluent was monitored sequentially for UV absorbance at 254 nm and radioactivity. Each sample was done in duplicate.

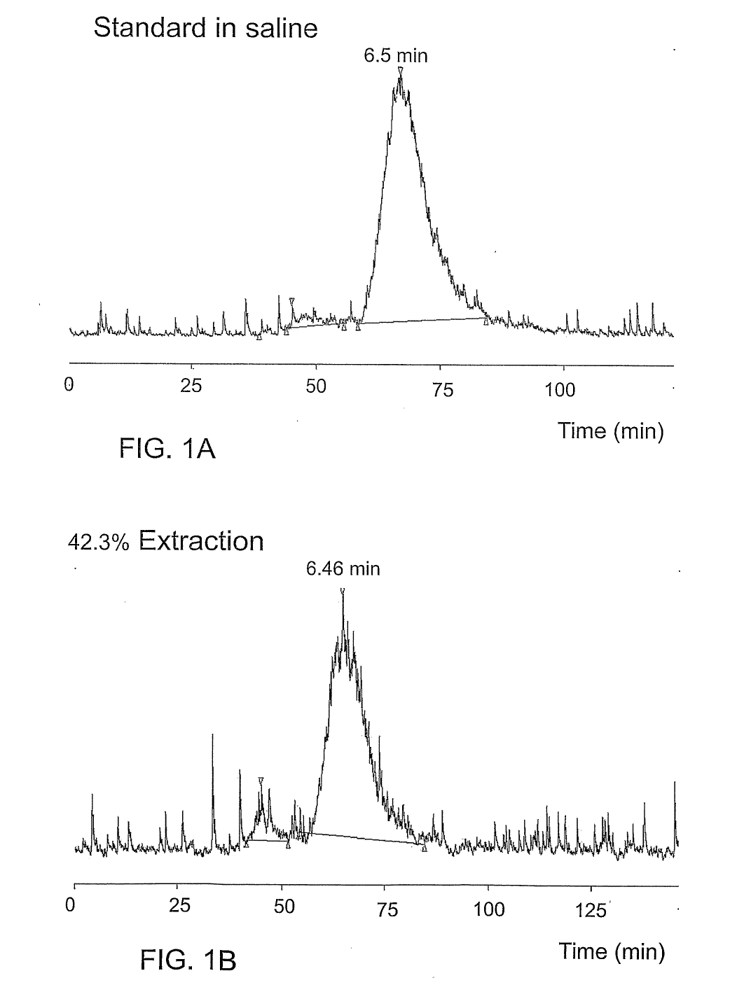

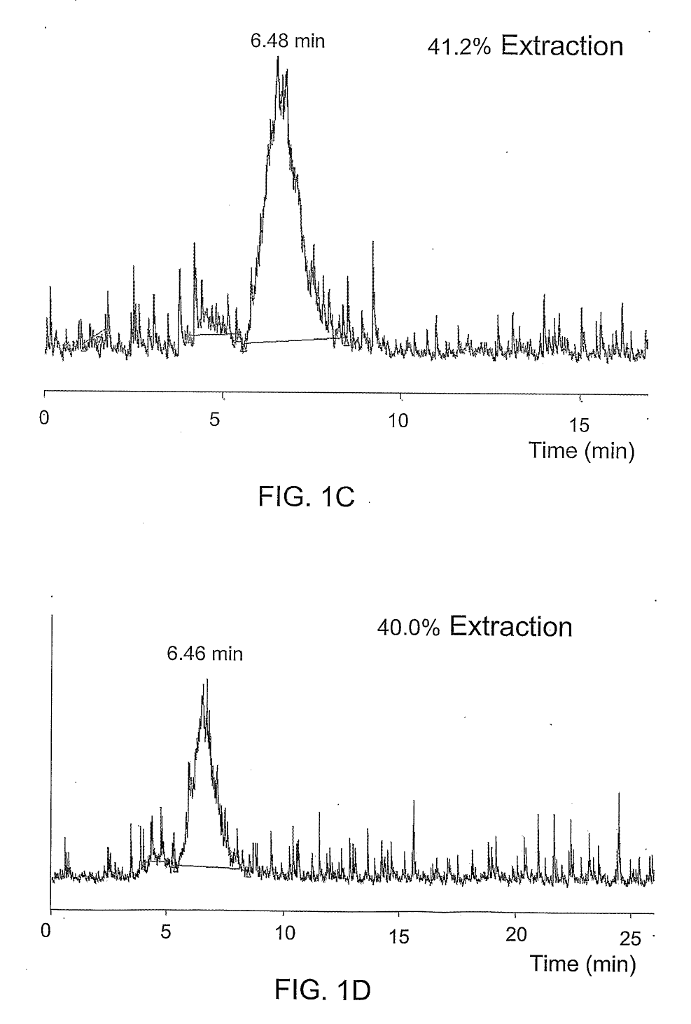

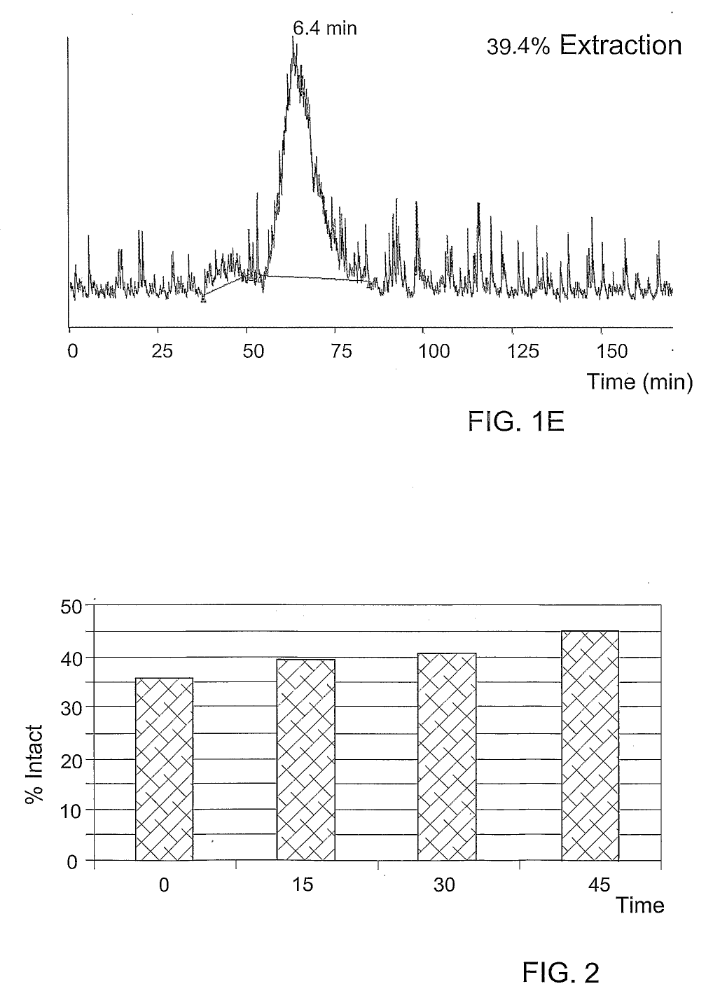

As shown in FIGS. 1A to 1E, the percent of extraction was 39-43% and no metabolites were evident.

The same procedure was done with the ¹¹C-labeled (R)-Compound 3 after incubation of 0, 15, 30 and 45 minutes.

Blood stability, which was measured by phosphor-imager, was done in the same procedure. The filtered samples were loaded on reverse phase TLC plates and exposed to the phosphor-imager plates for 1 hour.

In the case of (R)-Compound 2, the incubation in the human blood sample was 0, 15, 30 and 45 minutes. The percent of extraction of the compound was around 40%. The formation of metabolites was evaluated using phosphor-imager, and no metabolites were evident.

In-vivo blood stability experiments were performed for the ¹¹C-labeled (R)-Compound 3. The compound was injected into nude mice. Blood was sampled from the mice after 0, 15, 30 and 35 minutes. The labeled compound was extracted and evaluated using phosphor-imagers. No metabolites were evident as may be evident from FIG. 2.

### Example 8

### Biodistribution

The distribution of ¹¹C-labeled (R)-Compound 3 was measured in normal Lewis male rats which have been kept under SPF conditions. The labeled compound was injected through the tail vein of the rat. The rats were sacrificed 30, 60 and 90 minutes post injection, and prostate, blood, liver, muscle and kidney were removed and the activity in each organ was measured by gamma-counter (Tables 2A-D).

TABLE 2A
Biodistribution of ¹¹C-labeled (R)-Compound 3 in normal
Lewis male rats 30 minutes post injection.

| | Normal 30 min. | | | |
|---|---|---|---|---|
| % id/g | Rat 1 | Rat 2 | Av. | CV |
| Blood | 0.89 | 0.63 | 0.76 | 23.6 |
| Prostate | 1.67 | 1.27 | 1.47 | 19.3 |
| Liver | 4.26 | 3.67 | 3.96 | 10.5 |
| Muscle | 1.07 | 1.21 | 1.14 | 9.0 |
| Kidney | 15.57 | 9.29 | 12.43 | 35.7 |

TABLE 2B
Biodistribution of ¹¹C-labeled (R)-Compound 3 in normal
Lewis male rats 60 minutes post injection.

| | Normal 60 min. | | | |
|---|---|---|---|---|
| % id/g | Rat 1 | Rat 2 | Av. | CV |
| Blood | 0.54 | 0.41 | 0.48 | 19.9 |
| Prostate | 2.32 | 1.05 | 1.69 | 53.5 |
| Liver | 8.79 | 4.61 | 6.70 | 44.1 |
| Muscle | 1.02 | 0.68 | 0.85 | 27.7 |
| Kidney | 5.03 | 3.54 | 4.29 | 24.5 |

TABLE 2C
Biodistribution of ¹¹C-labeled (R)-Compound 3 in normal
Lewis male rats 60 minutes post injection.

| | Normal 60 min. | | | |
|---|---|---|---|---|
| % id/g | Rat 3 | Rat 4 | Av. | CV |
| Blood | 0.28 | 0.35 | 0.32 | 16.3 |
| Prostate | 1.13 | 1.18 | 1.16 | 3.0 |
| Liver | 5.30 | 5.14 | 5.22 | 2.2 |
| Muscle | 0.55 | 0.56 | 0.55 | 1.7 |
| Kidney | 2.48 | 2.54 | 2.51 | 1.6 |

TABLE 2D
Biodistribution of ¹¹C-labeled (R)-Compound 3 in normal
Lewis male rats 90 minutes post injection.

| | Normal 90 min. | | | |
|---|---|---|---|---|
| % id/g | Rat 1 | Rat 2 | Av. | CV |
| Blood | 0.3 | 0.42 | 0.36 | 22.6 |
| Prostate | 1.22 | 1.12 | 1.17 | 6.2 |
| Liver | 5.16 | 5.55 | 5.35 | 5.1 |
| Muscle | 0.48 | 0.57 | 0.53 | 11.7 |
| Kidney | 2.24 | 2.68 | 2.46 | 12.5 |

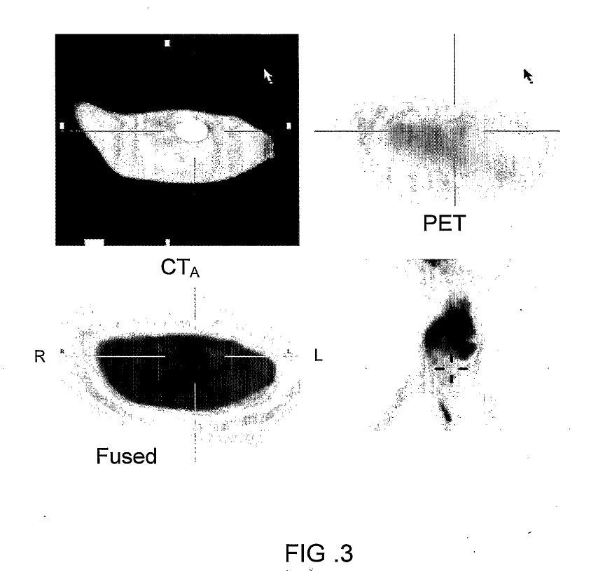

In some clinically used radio-tracers, such of FDG, activity is observed in the bladder, and mask the prostate. In order to verify that the injected compound did not accumulate in the bladder, and thus masked the prostate, the labeled compound was injected to normal Lewis male rats. The distribution of the compound was scanned by human PET-CT. Counter dye was injected to the animal 10 minutes prior to the injection of the labeled compound and the exact location of the bladder was detected by CT. No activity was observed in the bladder 40 minutes post injection (as shown in FIG. 3). In accordance with the biodistribution experiment, activity was measured in the metabolic organs.

### Example 9

### Antagonist Tests

PSA (Prostate Specific Antigen) is a secreted protein which is regulated by AR and is clinically used to detect prostate cancer in humans. LNCap cell lines were seeded in 24 wells (10⁵ cells/well) in 10% FCS RPMI supplemented with sodium-pyruvate, pen/strep, L-glutamine, testosterone and insulin and grown for 24 hours. Then, the cells were washed 3 times with cold PBS and incubated in 10% FCS red-phenol-free RPMI. Testosterone (100 nM), ¹⁸F-labeled (R)-Compound 3 (0.2, 0.5 and 1 μM) or both were added to the wells. Ethanol, which was used as the solvent of the testosterone and ¹⁸F-labeled (R)-Compound 3, was added to the control wells in the same concentration.

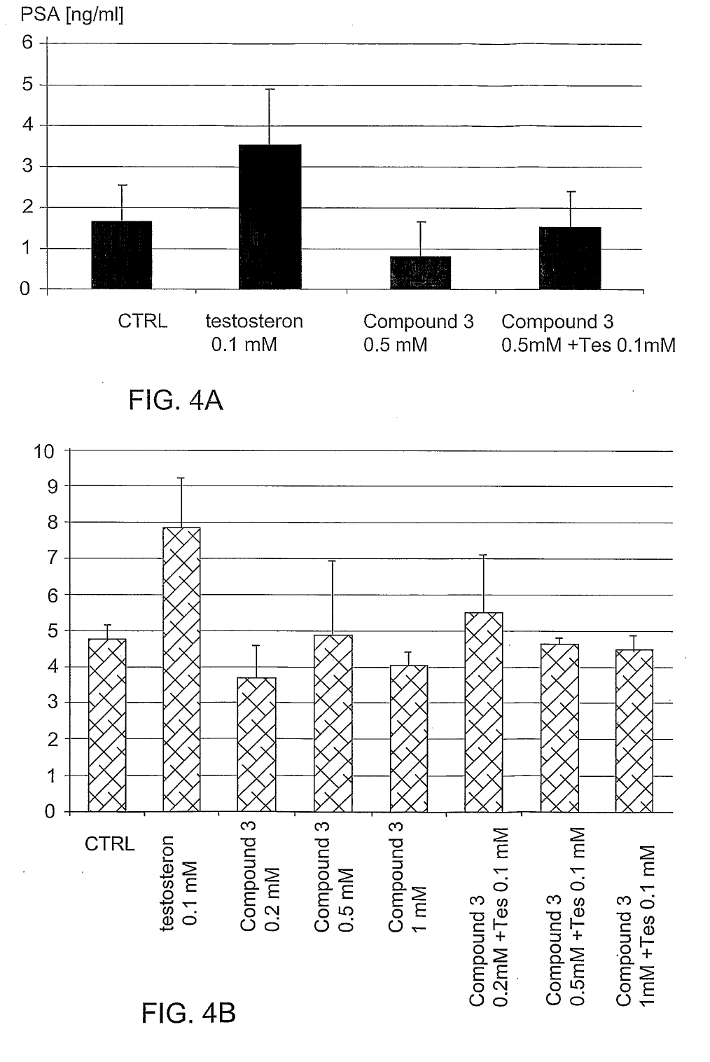

At 48 and 72 hours, a sample of the medium was taken and the PSA levels were measured by ELISA. Testosterone alone increased PSA secretion, in comparison to the control, while ¹⁸F-labeled (R)-Compound 3 reduced the PSA secretion. When put together, ¹⁸F-labeled (R)-Compound 3 significantly reduced the testosterone-induced secretion of PSA (P<0.01) (as shown in FIGS. 4A and B).

### Example 10

### Treatment Methodology

For LNCaP tumor experiments, treatments will begin 4-5 weeks after cell inoculation when measurable tumor volume is about 500 mm³. For each experiment, groups of six mice with comparable total tumor volumes will be either castrated or treated with a composition comprising one or more of the novel compounds or one or more of the compounds of the invention at 50 mg/kg/day. Compounds and reference drugs will be prepared at 10 mg/ml in a 0.3% solution of hydroxypropyl cellulose in saline, and mice will receive s.c. injections daily. Control and castrated mice will be treated with vehicle only. Treatments will last for 28 days, after which time the animals will be sacrificed and the blood will be collected. Tumors will be excised, weighed, and stored in liquid nitrogen for additional analysis. PSA levels will be measured in the serum using an Elisa kit.

### Schemes

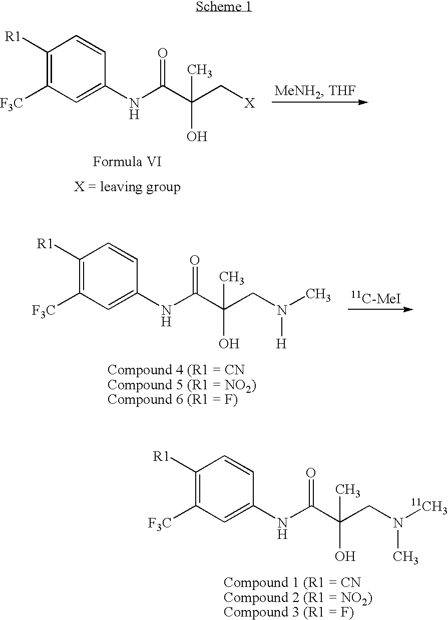

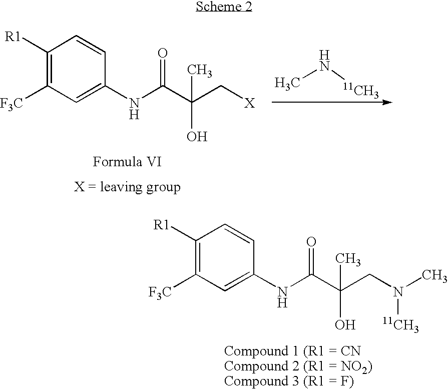

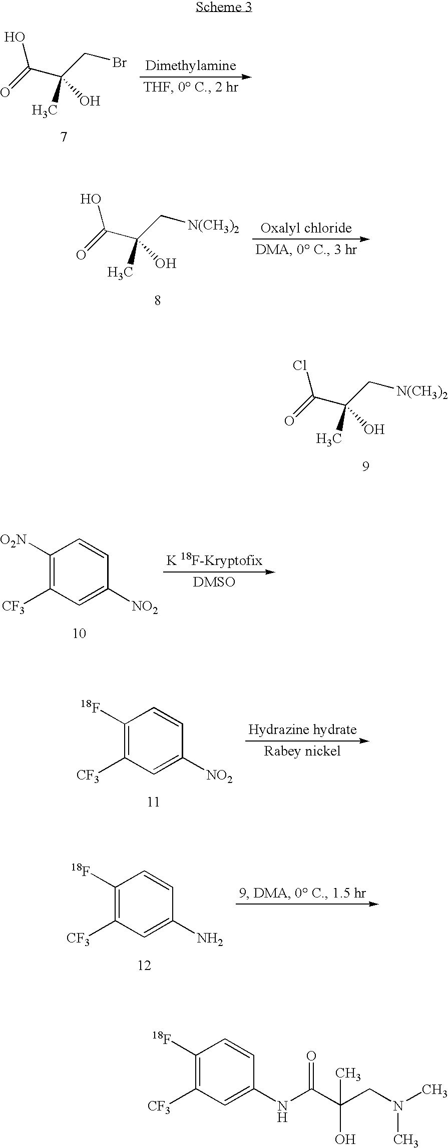

1-73. (canceled)

74. A compound of the general Formula I or a salt, ester, complex or enantiomer thereof: 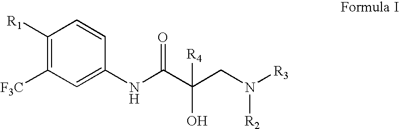
wherein
R1 is an electron withdrawing group;
R2 and R3, independently of each other, are each selected from H and a C1-C4 alkyl group being optionally substituted by at least one group selected from halogen, hydroxyl, alkyloxy, alkylthio, arylthio, alkoxy, alkylcarbonyl, carbonyl, alkoxycarbonyl, ester, amido, alkylamido, dialkylamido, aryl, benzyl, aryloxy, nitro, amino, alkyl or dialkylamino, carboxyl, or thio;
when R2 is H, R3 is a C1-C4 alkyl; and
R4 is a C1-C4 alkyl group being optionally substituted by at least one group selected from halogen, hydroxyl, alkoxy, alkylthio, alkylcarbonyl, carbonyl, alkoxycarbonyl, ester, amido, alkylamido, dialkylamido, aryl, benzyl, aryloxy, nitro, amino, alkyl or dialkylamino, carboxyl, and thio.

75. The compound according to claim 74, wherein said R1 is a radioisotope or comprising at least one radioisotope.

76. The compound according to claim 75, wherein said radioisotope is selected from ¹¹C, ¹⁸F, ¹⁵O, ¹³N, ¹²⁴I or ⁷⁶Br.

77. The compound according to claim 74, wherein R3 and/or R2, independently of each other is a C1-C4 group having at least one radioisotope.

78. The compound according to claim 77, wherein said radioisotope is selected from ¹¹C, ¹⁸F, ¹⁵O, ¹³N, ¹²⁴I or ⁷⁶Br.

79. The compound according to claim 74, wherein R2 and R3, independently of each other are each a C1-C4 alkyl being substituted.

80. The compound according to claim 74, when said R3 is —CH₃, said compound is of the Formula II:

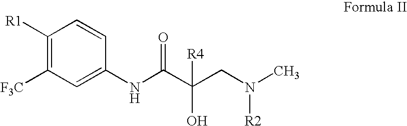

81. The compound according to claim 80, wherein R4 is an unsubstituted C1-C4 alkyl.

82. The compound according to claim 81, when said R4 is —CH₃, said compound is of the Formula III:

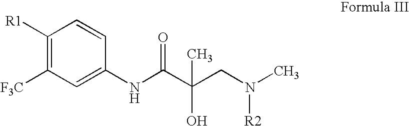

83. The compound according to claim 82, wherein said R2 is selected from H and a C1-C4 alkyl.

84. The compound according to claim 83, when R2 is H said compound is of the Formula IV:

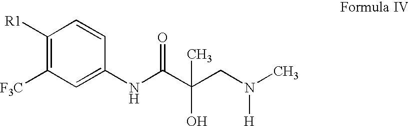

85. The compound according to claim 83, wherein R2 is C1-C4 alkyl group, being optionally substituted by at least one group selected from halogen, hydroxyl, alkoxy, alkylthio, alkoxy, alkylcarbonyl, carbonyl, alkoxycarbonyl, ester, amido, alkylamido, dialkylamido, aryl, benzyl, aryloxy, nitro, amino, alkyl or dialkylamino, carboxyl, and thio.

86. The compound according to claim 85, wherein said R2 is a linear, unsubstituted C1-C4 alkyl.

87. The compound according to claim 86, when said unsubstituted C1-C4 alkyl is methyl, said compound is of the Formula V:

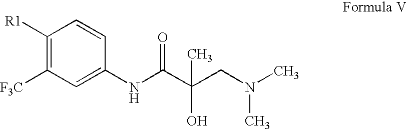

88. The compound according to claim 74, wherein said R1 is selected from —CF₃, —CN, —NO₂, —F, —I, —Br, —CHO, —COO-alkyl, or —CO-alkyl.

89. The compound according to claim 88, wherein said R1 is selected from —CF₃, —CN, —CHO, —COO-alkyl, or —CO-alkyl.

90. The compound according to claim 87, wherein said R1 is ¹¹C-labeled atom or group.

91. The compound according to claim 87, wherein said R1 is a group containing at least one halogen atom.

92. The compound according to claim 91, wherein said at least one halogen atom is a radioisotope.

93. The compound according to claim 92, wherein said radioisotope is one or more of ¹⁸F, ¹²⁴I, and ⁷⁶Br.

94. The compound according to claim 87, wherein said R1 is selected from —F, —NO₂ and —CN.

95. A compound herein designated Compound 1.

96. A compound herein designated Compound 2.

97. A compound herein designated Compound 3.

98. A compound herein designated Compound 4.

99. A compound herein designated Compound 5.

100. A compound herein designated Compound 6.

101. A radiolabeled form of the compound of claim 74.

102. The R-isomer of the compound of claim 74.

103. The S-isomer of the compound of claim 74.

104. A racemate of the compound of claim 74.

105. A method for the preparation of a compound of the general Formula I, said method comprising:
(i) condensing a suitable precursor molecule of the compound of Formula I with a mono-alkylamine, thereby affording a mono-alkylamine derivative of the compound of Formula I, and
(ii) reacting said mono-alkylamine derivative with an alkylating agent to thereby afford a compound of the general Formula I.

106. The method according to claim 105, wherein said suitable precursor molecule is of the Formula VI.

107. The method according to claim 105, wherein said mono-alkylamine is methylamine.

108. The method according to claim 105, wherein said alkylating agent is an alkylhalide.

109. The method according to claim 105, wherein said alkylhalide is selected from alkyliodide, alkylbromide and alkylchloride.

110. The method according to claim 109, wherein said alkylhalide is methylhalide.

111. The method according to claim 105, wherein one or both of said alkylamine and alkylating agent is radioactively labeled.

112. A method for the preparation of a compound of the general Formula I, said method comprising contacting a precursor molecule of the compound of Formula I with dialkylamine.

113. The method according to claim 112, wherein said dialklyamine is radioactively labeled.

114. A composition comprising at least one compound according to claim 74, or a compound prepared by the method of preparation of a compound of the general Formula I, said method comprising:
(i) condensing a suitable precursor molecule of the compound of Formula I with a mono-alkylamine, thereby affording a mono-alkylamine derivative of the compound of Formula I, and
(ii) reacting said mono-alkylamine derivative with an alkylating agent to thereby afford a compound of the general Formula I.

115. The composition according to claim 114 being a pharmaceutical composition or a diagnostic composition.

116. The composition according to claim 115, further comprising at least one pharmaceutically acceptable carrier, diluent or excipient.

117. The composition according to claim 115 for the treatment of at least one androgen-receptor (AR)-related pathology.

118. The composition according to claim 117, wherein said pathology is selected from one or more of the groups of (a) hormone related conditions; (b) androgen decline in females or males; (c) chronic muscular wasting; (d) androgen-related neoplastic diseases; (e) prostate cancer; (f) benign prostate hyperplasia; and (g) cancers of female sexual organs.

119. The composition according to claim 118, wherein said pathology is one or more of osteoporosis, periodontal disease, bone fracture, bone damage following bone reconstructive surgery, sarcopenia, frailty, aging skin, male hypogonadism, post-menopausal symptoms in women, female sexual dysfunction, atherosclerosis, hypercholesterolemia, hyperlipidemia, aplastic anemia and other hematopoietic disorders, pancreatic cancer, renal cancer, prostate cancer, breast cancer, hepatocellular carcinoma, glioma, meningioma, arthritis and joint repair.

120. The composition according to claim 119, wherein said pathology is a disease or disorder of the prostate.

121. The composition according to claim 120, wherein said disease or disorder of the prostate is prostate cancer.

122. A method for the treatment of androgen related disease or disorder, said method comprising administering to a subject in need thereof an effective amount of a composition comprising at least one compound according to claim 74, or a compound prepared by the method of preparation of a compound of the general Formula I, said method comprising:
(i) condensing a suitable precursor molecule of the compound of Formula I with a mono-alkylamine, thereby affording a mono-alkylamine derivative of the compound of Formula I, and
(ii) reacting said mono-alkylamine derivative with an alkylating agent to thereby afford a compound of the general Formula I.

123. The method according to claim 122, wherein said compound is not radioactively labeled.

124. The method according to claim 122, wherein said composition is co-administered to the subject with a further therapeutically useful substance or therapy.

125. The method according to claim 122, wherein said composition is administered to the subject in need thereof a method selected from oral, parenteral, paracanceral, transdermal, transmucosal, intravenous, intraperitonal, intramuscular, intracranial, intravaginal, intratumoral or via inhalation.

126. A method for binding a compound according to claim 74, or a compound prepared by the method of preparation of a compound of the general Formula I, said method comprising:
(i) condensing a suitable precursor molecule of the compound of Formula I with a mono-alkylamine, thereby affording a mono-alkylamine derivative of the compound of Formula I, and
(ii) reacting said mono-alkylamine derivative with an alkylating agent to thereby afford a compound of the general Formula I,
to an androgen receptor by contacting the receptor with said compound under conditions effective to cause said compound to bind the androgen receptor.

127. A radiopharmaceutical of the general Formula I or a salt, ester, complex or enantiomer thereof.

128. A method for imaging a tissue containing androgen receptor, said method comprising:
(i) contacting said tissue containing androgen receptor with an effective amount of a radiolabeled compound according to claim 74 under conditions which allow binding between said compound and the receptor;
(ii) subjecting the tissue to PET imaging; and
(iii) detecting regions of said tissue in which the radiolabeled compound has bound to an androgen receptor.

129. The method according to claim 128, wherein said tissue is an organism.

130. The method according to claim 129, wherein said organism is a human body.

131. A method according to claim 128, wherein said imaging is for the purpose of diagnosis, staging, re-staging or monitoring of the androgen related pathology.

132. A method for monitoring treatment of a subject having cancer cells or cells associated with an angiogenesis function disorder, said method comprises administering to said subject or a tissue a compound according to claim 74, and measuring the survival of the cells and/or the growth of the tumor.

133. The method according to claim 132, wherein said subject is suffering from a disease or disorder selected from leukemia, non-small cell lung cancer, colon cancer, CNS cancer, melanoma, ovarian cancer, breast cancer, renal cancer, hepatocellular carcinoma, glioma, meningioma and prostate cancer.
